(19) United States
(12) Patent Application Publication
(10) Pub. No.: US 2004/0081985 A1
(43) Pub. Date: Apr. 29, 2004

(54) Systems and method for examination of microarrays using scanning electron microscope

(75) Inventors: Paul C. Ciccolella, Menlo Park, CA (US); Maria A. Hozbor, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/615,560

(22) Filed: Jul. 8, 2003

Related U.S. Application Data
(60) Provisional application No. 60/395,520, filed on Jul. 12, 2002.

(51) Int. Cl. C12Q 1/68

(52) U.S. Cl. 435/6

(57) ABSTRACT

The present invention provides methods to detect biomolecules on a microarray using a scanning electron microscope. In one embodiment of the invention, errors in oligonucleotide synthesis during manufacturing of microarrays are detected by monitoring synthesis of control probes on the chips. In another embodiment, misalignment of features on the chip is determined. In yet another embodiment, the size, shape and edge definition of features on the chip is determined. In further embodiments, methods are provided for analyzing interactions between an oligonucleotide target and an oligonucleotide probe on a microarray and methods for testing conditions in a microarray manufacturing process.

### RELATED APPLICATIONS

This application claims the priority of U.S. provisional application serial No. 60/395,520, filed on Jul. 12, 2002. The '520 application is incorporated herein by reference.

This invention relates generally to the field of manufacturing of microarrays, and, in particular, to the use of scanning electron microscopy in the detection of biomolecules on a microarray, analysis of microarrays for defects, evaluation of test conditions in manufacturing, and the feature quality of microarrays.

### BACKGROUND OF THE INVENTION

Microarrays are useful in a variety of screening techniques for obtaining information about either the probes or the target molecules. For example, a library of peptides can be used as probes to screen for drugs. The peptides can be exposed to a receptor, and those probes that bind to the receptor can be identified.

Microarrays wherein the probes are oligonucleotides (“DNA chips”) show particular promise. Arrays of nucleic acid probes can be used to extract sequence information from nucleic acid samples. The samples are exposed to the probes under conditions that allow hybridization. The arrays are then scanned to determine to which probes the sample molecules have hybridized. One can obtain sequence information by selective tiling of the probes with particular sequences on the arrays, and using algorithms to compare patterns of hybridization and non-hybridization. This method is useful for sequencing nucleic acids. It is also useful in diagnostic screening for genetic diseases or for the presence of a particular pathogen or a strain of pathogen.

The scaled-up manufacturing of oligonucleotide arrays requires application of quality control standards both for determining the quality of chips under current manufacturing conditions and for identifying optimal conditions for their manufacture. Quality control, of course, is not limited to manufacture of chips, but also to the conditions under which they are stored, transported and, ultimately, used.

### SUMMARY OF THE INVENTION

The present invention provides methods to detect biomolecules on a microarray using a scanning electron microscope. In one embodiment of the invention, errors in oligonucleotide synthesis during manufacturing of microarrays are detected by monitoring synthesis of control probes on the chips. In another embodiment, misalignment of features on the chip is determined. In yet another embodiment, the size, shape and edge definition of features on the chip is determined. In further embodiments, methods are provided for analyzing interactions such as hybridization between an oligonucleotide target and an oligonucleotide probe on a microarray and methods for testing conditions in a microarray manufacturing process.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1. A scanning electron microscopy image showing the detection of oligonucleotides on a microarray.

FIG. 2. A scanning electron microscopy image showing different synthesis events. In one feature, there is no explosure and no biomolecules are synthesized. In a second feature, there is double exposure and darker feature is observed. The normal features represent biomolecules synthesized upon single exposure.

FIG. 3. A scanning electron microscopy image showing misalignment.

FIG. 4. A scanning electron microscopy image showing a set of Vernier scales designed to detect misalignment.

FIG. 5. A scanning electron microscopy image showing one-micron resolution lines.

FIG. 6. A high magnification and SEM image of gold nanospheres on glass.

FIG. 7. An SEM image of an unhybridrized GeneChip® DNA microarray.

FIG. 8. An SEM image of photolithographic resolution lines on an unhybridized GeneChip® DNA microarray.

FIG. 9. A low magnification SEM image of streptavidin-gold labeled target in a complex background.

FIG. 10. A low magnification SEM image of streptavidin-gold labeled target in complex background.

FIG. 11. An SEM image of photolithographic resolution lines labeled with streptavidin-gold.

FIG. 12. A back scattered electron image of checkerboard patterned streptavidin-gold labeled target.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example hereinbelow. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, Biochemistry, (W H Freeman), Gait, “Oligonucleotide Synthesis: A Practical Approach” 1984, IRL Press, London, all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US 01/04285, and in U.S. patent applications Ser. Nos. 09/501,099 and 09/122,216 which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefor are shown in U.S. Ser. No. 10/013,598, and U.S. Pat. Nos. 5,856,092, 6,300,063, 5,858,659, 6,284,460 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. For example, see the patents in the gene expression, profiling, genotyping and other use patents above, as well as U.S. Ser. No. 09/854,317, Wu and Wallace, Genomics 4, 560 (1989), Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), Guatelli et al., Proc. Nat. Acad. Sci. USA, 87, 1874 (1990), WO 88/10315, WO 90/06995, and 6,361,947.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625 and in PCT Application PCT/US99/06097 (published as WO99/47964), each of which also is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over the internet. See provisional application No. 60/349,546.

In scanning electron microscopy (SEM) an electron beam is focused into a small probe and is rastered acrossed the surface of a specimen. Several interactions with the sample that result in the emission of electrons or photons occur as the electrons penetrate the surface. These emitted particles can be collected with the appropriate detector to yield valuable information about the material.

By scanning an electron probe across a specimen, the secondary electrons produced yield high resolution images of the morphology and topography of a specimen with great depth of field from a low to a very high magnification. Maps of atomic number of the sample can also result from analyzing the backscatter electron signal and compositional analysis of a material can be obtained by monitoring x-rays produced by the electron-sample interaction. A scanning electron microscope consists of an electron source, an electron column, a probe forming system, alignment coils, lenses, aperture assembly, astigmatism correction, scan coils, specimen holder, vacuum system, detection system and associated electronics.

In one embodiment of the invention, using a Hitachi S-4700 SEM (Hitachi High-Technologies American Inc., Pleasanton, Calif.), one can view a microarray without a coating under the following conditions: using analysis mode with accelerating voltages ranging from 500 eV to 2 keV, a large spot size by changing either the aperture and/or condenser lens settings, using a high emission current ranging from 20 to 50 μA, and using a upper detector.

In another embodiment of the invention, a coating can be used to reduce charging. One of skill in the art will appreciate that many types of coatings may be selected to reduce charging. One example of coating is a gold/palladium coating with thickness ranging from 1 to 10 nm, preferably from 1.5 to 3 nm. In a further embodiment, using a Hitachi S-4700 SEM, one can view a microarray with coating by using analysis mode with accelerating voltages ranging from 3 keV to 10 keV, a large spot size by changing either the aperture and/or condenser lens settings, using a high emission current ranging from 20 to 50 μA, and using the upper detector. It is understood that one of skill in the art will appreciate ways of viewing a microarray with or without coatings using a scanning electron microscope under appropriate conditions.

In one aspect of the invention, biomolecules on a microarray are detected by scanning the microarray with a scanning electron microscope. The term “biomolecule” as used herein refers to a polymeric form of biological or chemical moieties. Representative biomolecules include, but are not limited to, nucleic acids, oligonucleotides, polynucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. A preferred biomolecule is a nucleic acid, which includes oligonucleotides and polynucleotides. A preferred nucleic acid is formed from 10 to 50 nucleotide bases. Another preferred nucleic acid has 50 to 1,000 nucleotide bases. The nucleic acid may be a PCR product, PCR primer, or nucleic acid duplex, to list a few examples. In this invention, the terms nucleic acid, oligonucleotide and polynucleotide are used interchangeably to one another.

I. Microarray Manufacturing Processes

As used herein, “spatially directed oligonucleotide synthesis” refers to any method of directing the synthesis of an oligonucleotide to a specific location on a substrate. Methods for spatially directed oligonucleotide synthesis include, without limitation, light-directed oligonucleotide synthesis, microlithography, application by ink jet, microchannel deposition to specific locations and sequestration with physical barriers. In general these methods involve generating active sites, usually by removing protective groups; and coupling to the active site a nucleotide which, itself, optionally has a protected active site if further nucleotide coupling is desired.

The term “oligonucleotide” or “polynucleotide” refers to a single- or double-stranded deoxyribonucleic acid (DNA) or ribonucleic acid (RNA) polymer containing deoxyribonucleotides or ribonucleotides or analogs of either. Oligonucleotides can be naturally occurring or synthetic, but are typically prepared by synthetic means. Suitable oligonucleotides may be prepared by the phosphoramidite method described by Beaucage et al., 1981, Tetr. Lett. 22: 1859-1862, or by the triester method, according to Matteucci et al., 1981, J. Am Chem. Soc. 103: 3185, or other methods, such as by using commercially available, automated oligonucleotide synthesizers. Polynucleotides of the present invention include sequences of DNA or RNA which may be isolated from natural sources, recombinantly produced or artificially synthesized and mimetics thereof. A further example of an oligonucleotide or a polynucleotide of the present invention may be peptide nucleic acid (PNA).

Oligonucleotide arrays can be synthesized at specific locations by light-directed oligonucleotide and polynucleotide synthesis. The pioneering techniques of this method are disclosed in U.S. Pat. No. 5,143,854; PCT WO 92/10092; PCT WO 90/15070; U.S. Pat. Nos. 5,571,639, 5,744,305; and 5,968,750, incorporated herein by reference for all purposes. The basic strategy of this process is described in U.S. Pat. Nos. 5,424,186 and 6,307,042. The surface of a solid support modified with linkers and photolabile protecting groups is illuminated through a photolithographic mask, yielding reactive hydroxyl groups in the illuminated regions. A 3′-O-phosphoramidite-activated deoxynucleoside (protected at the 5′-hydroxyl with a photolabile group) is then presented to the surface and coupling occurs at sites that were exposed to light. Following the optional capping of unreacted active sites and oxidation, the substrate is rinsed and the surface is illuminated through a second mask, to expose additional hydroxyl groups for coupling to the linker. A second 5′-protected, 3′-O-phosphoramidite-activated deoxynucleoside is presented to the surface. The selective photodeprotection and coupling cycles are repeated until the desired set of products is obtained. Photolabile groups are then optionally removed and the sequence is, thereafter, optionally capped. Side chain protective groups, if present, are also removed. Since photolithography is used, the process can be miniaturized to generate high-density arrays of oligonucleotide probes. Furthermore, the sequence of the oligonucleotides at each site is known.

This general process can be modified. For example, the nucleotides can be natural nucleotides, chemically modified nucleotides or nucleotide analogs, as long as they have activated hydroxyl groups compatible with the linking chemistry. The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, which results in a free 5′ end.

The general process of removing protective groups by exposure to -light, coupling nucleotides (optionally competent for further coupling) to the exposed active sites, and optionally capping unreacted sites is referred to herein as “light-directed nucleotide coupling.”

Another method of spatially directed oligonucleotide synthesis involves mechanically directing nucleotides to specific locations on a substrate for coupling, for example, by ink jet technology. Ink jets currently can apply material to specific locations in areas as small as 200 square microns in diameter. (See, e.g., U.S. Pat. No. 5,599,695, incorporated herein by reference.)

Another method of spatially directed oligonucleotide synthesis involves directing nucleotides to specific locations on a substrate for coupling by the use of microchannel devices. Microchannel devices are described in more detail in International application WO 93/09668, incorporated herein by reference.

Another method of spatially directed oligonucleotide synthesis involves directing nucleotides to specific locations on a substrate for coupling by the use of physical barriers. In this method, a physical barrier is applied to the surface such that only selected regions are exposed to the conditions during polymer chain extension. For example, the surface of a chip may be coated with a material that can be removed upon exposure to light. After exposing a particular area to light, the material is removed, exposing the surface of the chip for nucleotide coupling. The exposed surface in this area can be exposed to the nucleotide, while the other areas or regions of the chip are protected. Then, the exposed area is re-covered, and protected from subsequent conditions until re-exposure. See, e.g., WO 93/09668, incorporated herein by reference.

Methods of spatially directed synthesis can be used for creating arrays of other kinds of molecules as well, and these arrays also can be tested by the methods of this invention. For example, using the strategies described above, spatially patterned arrays can be made of any molecules whose synthesis involves sequential addition of units. This includes polymers composed of a series of attached units and molecules bearing a common skeleton to which various functional groups are added. Such polymers include, for example, both linear and cyclic polymers of nucleic acids, polysaccharides, phospholipids, and peptides having either .alpha.-, .beta.-, or .omega.-amino acids, heteropolymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers which will be apparent to anyone skilled in the art. Molecules bearing a common skeleton include benzodiazepines and other small molecules, such as described in U.S. Pat. No. 5,288,514.

II. Detection of Defects on a Microarray

Oligonucleotide microarrays are typically fabricated, in part, by synthesizing oligonucleotides on selected positions of a wafer substrate (features). The escalating requirement for high density performance requires design features of less than twenty microns, preferably less than ten microns, more preferably less than five microns, even more preferably less than one micron. The reduction of design features challenges the limitations of conventional microarray manufacturing techniques as well as methodologies for detection and characterization of microarrays and the defects contained therein.

One factor that affects manufacturing yield is the presence of defects on the microarrays from the manufacturing process. Defects can take various forms, such as, for example, synthesis errors, misalignments, scratches, and particles. Undetected defects can often lead to failure of a microarray that is made from the wafer.

Some in-process inspection and review is normally performed to detect and to classify defects that are detected on the wafer during the manufacturing process. Classification of defects on the wafer involves, among other things, the ability to extract accurate information such as defect size, shape, and boundary in order to identify the sources of the defects. This operation requires high resolution imaging. As features on the wafers become smaller, however, the size of the defects that can affect production yield also become smaller. Accordingly, the SEM can be used for higher resolution systems for defect classification. The SEM is capable of resolving defects on an array with a size of less than a micron and it can be useful for reducing defects in a microarray manufacturing process, more specifically for optimizing a lithographic process to reduce defects and to qualify the optimized lithographic process for production.

According to one aspect of the present invention, a method of reducing defects in a microarray manufacturing process comprises forming a pattern on a first wafer using the microarray manufacturing process according to a prescribed processing specification, inspecting the pattern on the first wafer to detect a first defect, developing an alternative processing specification relative to the prescribed processing specification based on the first defect, forming the pattern on a wafer using the microarray manufacturing process according to the alternative processing specification, comparing respective characteristics of the patterns on the first and second wafers, and changing the manufacturing process to include the alternative processing specification based on the comparing step. The formation of the pattern on the first wafer using the microarray manufacturing process according to the prescribed processing specification enables precise analysis of the prescribed processing specification forming the pattern, without introducing additional variables that may otherwise be present during manufacturing of a microarray product. In addition, the inspecting of the pattern on the first wafer to detect a first defect may be implemented as a short loop test, where defect causes related to the prescribed processing specification can be efficiently identified, including both killer defects directly affecting yield and non-killer defects. The comparison of the respective characteristics of the patterns on the first and second wafers also enables the alternative processing specification to be qualified relative to the prescribed processing specification in an efficient manner.

The SEM can also be used for detecting random defects occurring during photolithography processing, and for monitoring the random defects to optimize the lithographic process.

These and other uses of the SEM are shown in the present invention, where a pattern formed on a wafer using a microarray manufacturing process simulating a prescribed processing specification and the array is inspected for defects. The detected defects are then classified, enabling generation of an alternative processing specification. The alternative processing specification is then tested by synthesis of oligonucleotides on different wafers using the alternative processing specification, and then analyzing the success on the different wafers relative to the prescribed processing specification. The testing thus enables qualification of the alternative processing specification for production of microarray products.

III. Testing Processes in Microarray Manufacturing

In making a microarray, the substrate and its surface preferably form a rigid support on which the sample can be formed. See the array patents above such as U.S. Pat. No. 5,143,854, for exemplary supports. The substrate and its surface are also chosen to provide appropriate light-absorbing characteristics. For instance, the substrate may be functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, polycarbonate, or combinations thereof. Other substrate materials will be readily apparent to those skilled in the art upon review of this disclosure. In a preferred embodiment the substrate is flat glass or silica.

Surfaces on the solid substrate usually, though not always, are composed of the same material as the substrate. Thus, the surface may be composed of any of a wide variety of materials, for example, polymers, plastics, resins, polysaccharides, silica or silica-based materials, carbon, metals, inorganic glasses, membranes, or any of the above-listed substrate materials. In one embodiment, the surface will be optically transparent and will have surface Si—OH functionalities, such as those found on silica surfaces.

Preferably, oligonucleotides are arrayed on a chip in addressable rows and columns. Technologies already have been developed to read information from such arrays. The amount of information that can be stored on each chip depends on the lithographic density which is used to synthesize the wafer. For example, if each feature size is about 100 microns on a side, each chip can have about 10,000 probe addresses in a 1 cm² area. For further example, if each feature size is about 10 microns on a side, each chip can have about 1,000,000 probe addresses in a 1 cm² area.

A general method of this invention is directed to determining the extent to which a test condition affects the appearance of a feature an oligonucleotide array produced by spatially directed oligonucleotide synthesis. This method involves providing a substrate having a surface with linkers having an active site for oligonucleotide synthesis. An ensemble of sequence-specific oligonucleotides is synthesized on the substrate by spatially directed oligonucleotide synthesis. The oligonucleotides can be provided with active sites for attaching a detectable label. The area is exposed to the test condition. The Scanning Electron Microscope (SEM) is capable of detecting and resolving such features.

The methods of this invention are very versatile. An array can have several ensembles of different sequence-specific oligonucleotides. Within any one ensemble, several sub-areas can be exposed to different test conditions. Thus, several different ensembles can be exposed to several different test conditions on a single array. The oligonucleotide array can be exposed to one or more test conditions throughout the microarray production process, or at specific times. The test conditions can change during the production process. Exposing different ensembles to the same condition is useful to test the effect of a condition on particular oligonucleotide sequences. Exposing ensembles of oligonucleotides to different conditions assists in identifying the effect of a condition on the manufacturing process.

The conditions to be tested by the methods of this invention are at the discretion of the practitioner. However, usually the practitioner will select conditions to be tested for the manufacturing process. These can include, for example, light, temperature, humidity, mechanical stress, reagents used in the synthesis, storage conditions, transportation conditions and operation conditions.

Many parameters involved with the manufacturing of oligonucleotide arrays can be tested. Of course, conditions can be applied to specific locations, or specific oligonucleotides can be synthesized at particular locations and the entire substrate can be subject to a test condition to determine the effect at each area.

The effect of the testing conditions on the manufacturing process can then be evaluated by inspecting the features on the array using the scanning electron microscope. The microarray manufacturing process is thus optimized.

According to one aspect of the present invention, a method of testing conditions in a microarray manufacturing process comprises manufacturing a microarray on said first wafer using the microarray manufacturing process according to a prescribed processing specification, inspecting the pattern on the first wafer to detect the effect of a condition, developing an alternative processing specification relative to the prescribed processing specification based on the first condition, forming the pattern on a second wafer using the microarray manufacturing process according to the alternative processing specification, comparing respective characteristics of the patterns on the first and second silicon wafers, and changing the lithographic process, chemistry process, or other manufacturing processes to include the alternative processing specification based on the comparing step. The formation of the pattern on the first wafer using the microarray manufacturing process according to the prescribed processing specification enables precise analysis of the prescribed processing specification forming the pattern, without introducing additional variables that may otherwise be present during manufacturing of a microarray product.

IV. Other Applications

Scanning electron microscopy is also useful as a navigation tool on a surface patterned with oligonucleotides and a defect finding tool on a surface patterned with oligonucleotides. SEM may find utilities to detect missing steps in synthesis, to determine the shape and position of an oligonucleotide feature produced during synthesis, and to determine the length of oligonucleotide probes patterned on a surface. SEM is also used as an in-process tool for detecting the presence of partial oligonucleotide sequences and for detecting whether a probe has been hybridized to a target.

Scanning electron microscopy is useful to detect oligonucleotides hybridized to oligonucleotide probes on a microarray. In one embodiment, a method is disclosed to analyze interactions between an oligonucleotide target and an oligonucleotide probe on a microarray, comprising exposing a oligonucleotide probe on a microarray to a plurality of oligonucleotide targets under a hybridization condition, then scanning the microarray with a scanning electron microscope; and finally detecting the oligonucleotide targets binding to the oligonucleotide probe on the microarray. In another embodiment, the microarray is synthesized by light directed oligonucleotide syntheses, then exposed to nucleic acid targets under a hybridization condition and scanned with a scanning electron microscope to detect the targets binding to the probes on the microarray. In yet another embodiment, the oligonucleotide target is labeled with a heavy atom, such as a colloidal gold or palladium. Such a heavy atom can be detected using either a secondary electron detector or a backscattered electron detector.

Oligonucleotides may be hybridized to probes on a microarray under a hybridization condition. One of skill in the art will appreciate that hybridization conditions may be selected to provide any degree of stringency. In a preferred embodiment, hybridization is performed at low stringency in this case in 6×SSPE-T at about 40° C. to about 50° C. (0.005% Triton X-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPE-T at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatches controls, etc.).

Microarrays are then scanned using a scanning electron microscope to detect the hybridized oligonucleotides. The target oligonucleotides may be labeled with electron scattering atoms such as heavy atoms to enhance detection of target oligonucleotides. In one embodiment, the heavy atoms are colloidal gold and palladium. In another embodiment, the heavy atom is detected using a backscattered electron detector.

In addition to using scanning electron microscopy for the detection of biomolecules on a microarray, one of skill in the art will appreciate using other sources to provide beams of electrons for the detection of biomolecules on microarrays.

Scanning electron microscopy (SEM) has long been a employed in the semiconductor and other high technology fields. SEM has also been used more recently in the life science field to study DNA (Younghusband, H. B.; Inman, R. B.; Annual Review of Biochemistry, 43, 605 (1974)).

The use of colloidal gold in life science applications at SEM is known. (R. Hermann, P. Wlather, M. Muller Histochem Cell Biol 106, 31 (1996)). Uses include labeling of cells and labeling of DNA and RNA for high resolution imaging (Erlandsen, S. L.; Macechko, P. T.; Frethem, C.; Scanning Microscopy, 13, 43 (1999)). Methods of detection using gold nanospheres labeled with oligonucleotides are also well established. (Taton, T. A.; Lu, G.; Mirkin, C. A. J. Am. Chem. Soc. 123, 5164 (2001)).

Others have used techniques such as Surface Plasmon Resonance (SPR) and colloidal gold to detect DNA hybridization. (He, L.; Musick, M. D.; Nicewarner, S. R.; SWalinas, F. G.; Benkovic, S. J.; Natan, M. J.; Keating, C. D. J. Am Chem. Soc 122, 9071 (2000)).

The present invention contemplates as a preferred embodiment using SEM on DNA microarrays without staining or hybridization, allowing the use of the subtle contrast mechanism present in each feature for defect analysis, navigation for defect location, and to monitor and characterize the manufacturing process.

Also contemplated by the present invention is the use of gold nanospheres in conjunction with SEM and high density DNA arrays to characterize and define feature quality and optimize manufacturing using hybridization detection. The combination of the high resolution of the SEM and specificity of the streptavidin coated gold nanospheres as disclosed with respect to the instant invention make an excellent research tool for characterizing DNA microarrays.

Also contemplated by the present invention is the use of intercalating or other types of molecules that bind to DNA and RNA such as psoralen compounds, ethidium bromide compounds, or cis-platinum compounds. These intercalators are available as compounds with biotin and could be applied to microarrays. The molecules will bind to short sequences of DNA and RNA, such that hybridization with a complementary target is not necessary. These biotin complexes can then be labeled with streptavidin coated gold nanospheres and detected using SEM.

Additional objects, advantages and novel features of the invention will be set forth in part in the description which follows, and in part will become apparent to those skilled in the art upon examination of the following or may be learned by practice of the invention.

The invention will be further understood by the following non-limiting examples.

### EXAMPLES

### Example 1

### SEM for Detection of Control Probes

The preferred way to enhance the contrast of the control probes to view the chip before any hybridization and without any sputtered coating. The Analysis Mode SEM setting provides the most current and thus best contrast enhancement. Low voltages, i.e. 1 kV, are necessary to prevent charging of the sample. The upper detector, optimized to detect SE1 electrons, is better than either the lower detector or a mix of upper and lower detectors. To avoid a “shadow” gradient, the working distance must be optimized for each sample. The emission current should be between 20 μA and 40 μA. The higher the emission current the more likely the sample is to charge, therefore, uses the maximum current without adverse affects of charging.

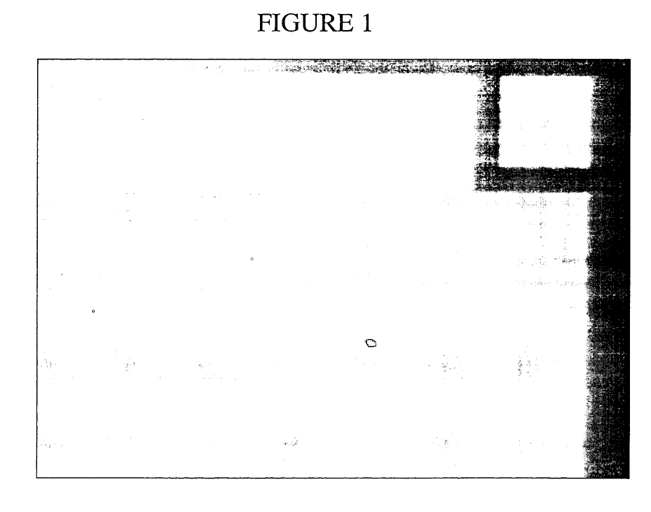

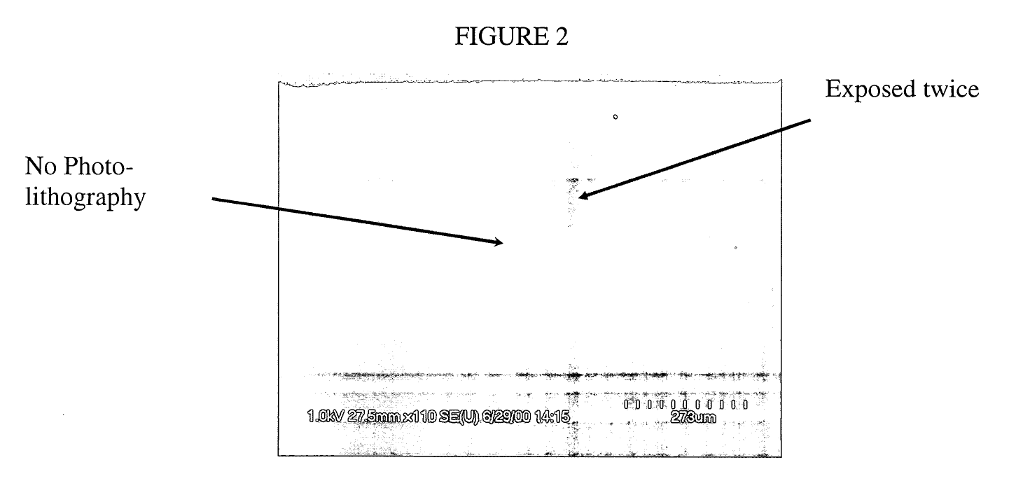

The SEM can clearly distinguish the bases from background. In FIG. 1, a scanning electron microscopy image showing the detection of oligonucleotides on a microarray is shown. In FIG. 2, a mask was skipped and another mask was used twice. It can be clearly seen with SEM (also seen clearly with fluorescence staining) that the area where a mask should have been exposed is blank and the square for the other masks are slightly darker, indicating more DNA present.

### Example 2

### SEM to Detect Photolithographic Misalignments

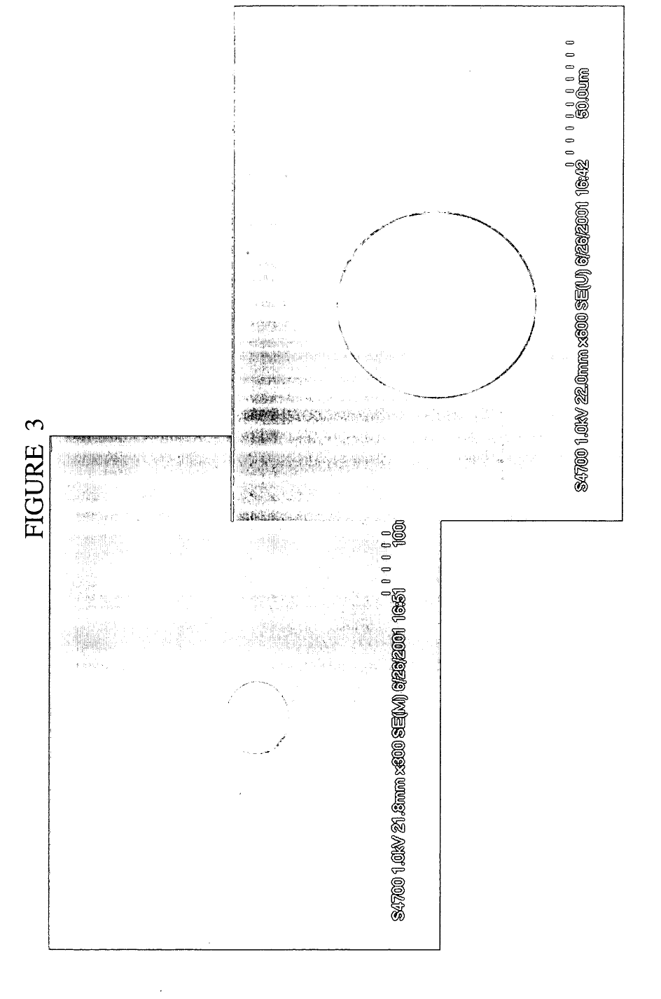

It has also been possible to use the control probes as in Example 1 to detect the presence of photolithographic misalignments. The SEM can detect very minor misalignments. FIG. 3 shows the square associated with step 30 is shifted in the x and y directions. The shift is about 3 microns in each direction.

### Example 3

### SEM for Examination of Mask Design

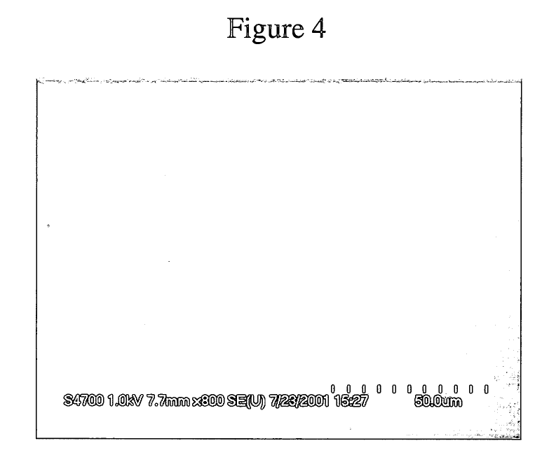

SEM analysis has also been used to examine a development mask design. Contained in this design are a set of vernier scales designed to detect misalignment and a series of resolution lines ranging from 50 microns to 1 micron in size. FIG. 4 shows the SEM image of the vernier scales.

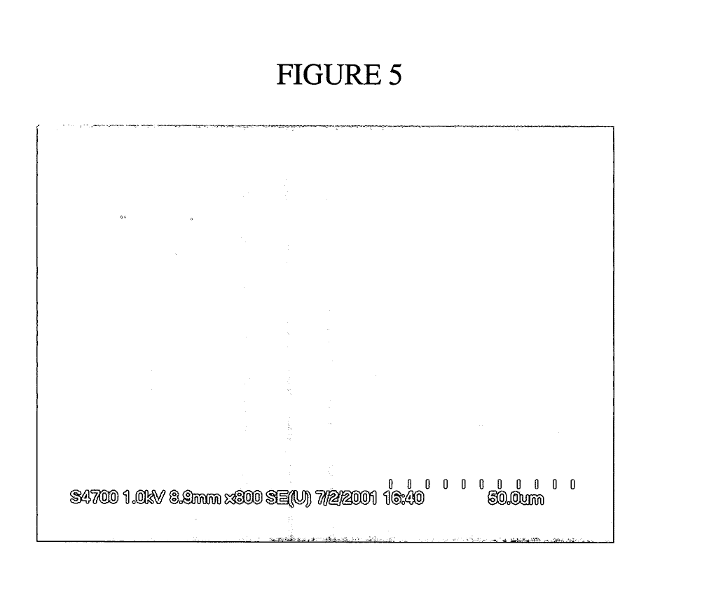

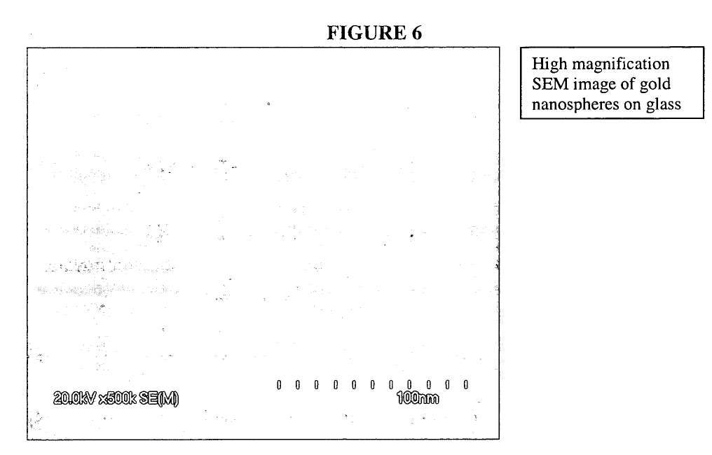

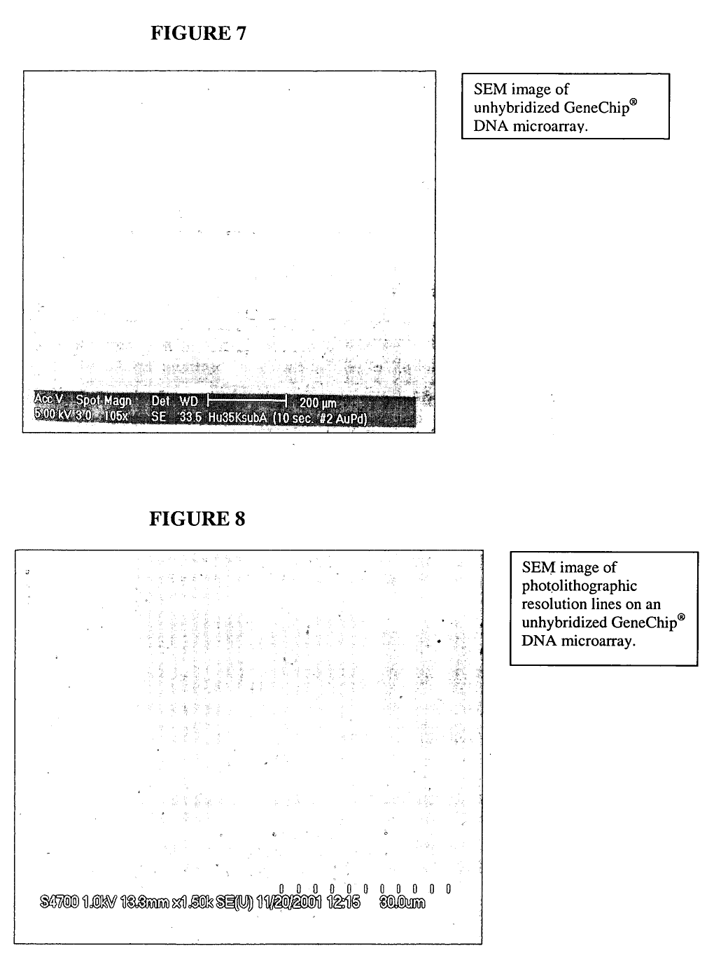

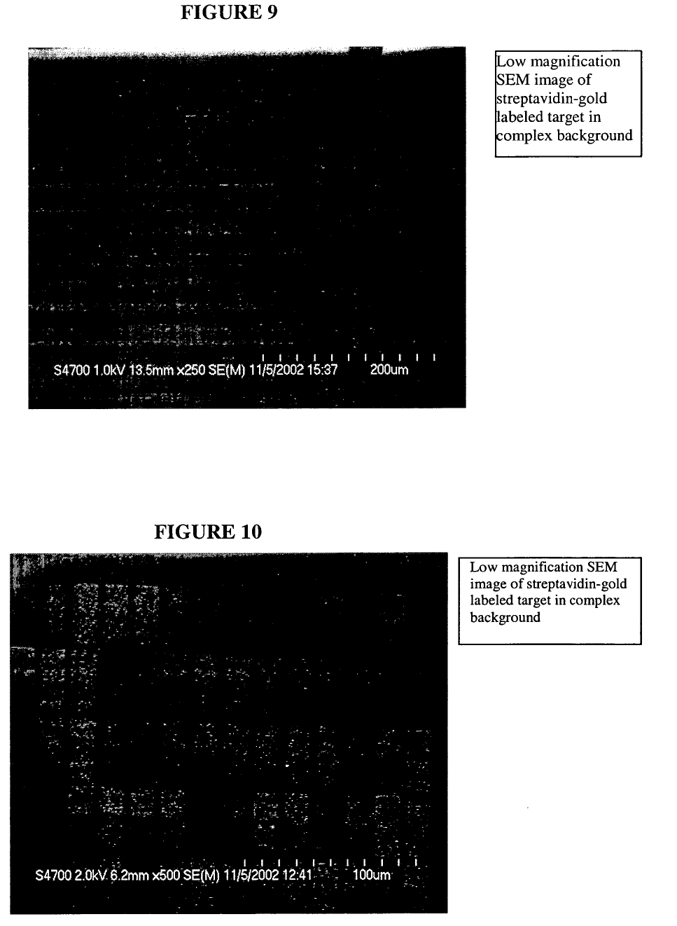

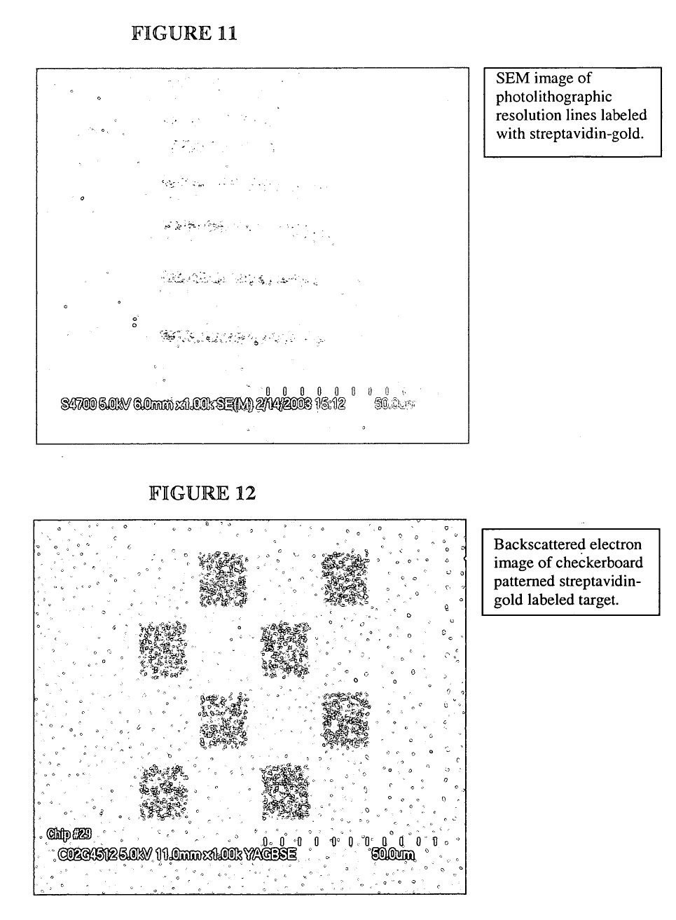

The SEM has also been used to determine the resolution using the current photolithography techniques by imaging the resolution lines. By analyzing the resolution lines, the width of the lines and the space between two lines (which can be a quantitatively defined as the resolution) can be determined for the steps when these features are printed. FIG. 5 presents a SEM image showing that the 1 micron spacing can be resolved.

### Example 4

### SEM of Gold Labeled Arrays

Materials:

Distilled water and Acetylated Bovine Serum Albumin (BSA) solution (50 mg/mL) were obtained from Invitrogen Life Technologies. 5 M NaCl, RNase-free, DNase-free, was from Ambion. MES Free Acid Monohydate SigmaUltra, MES Sodium Salt, and EDTA Disodium Salt were purchased from Sigma-Aldrich. 10% surface-Amps20 (Tween 20) was from Pierce Chemical. 20×SSPE (3M NaCl, 0.2 M NaH₂PO₄, 0.02 M EDTA) was purchased from Bio Whittaker. 12× MES stock(1.22 M MES, 0.89 M [Na+], Stringent Wash Buffer (100 mM MES, 0.1M [Na⁺], −0.01% Tween 20), Non-stringent wash buffer (6× SSPE, 0.01% Tween 20 and 2× MES buffer (100 mM MES, 1 M [Na⁺], 0.05% Tween 20) were prepared following the GeneChip® Expression Analysis Technical manual provided by Affymetrix. Nanogold®-Streptavidin conjugate and GoldEnhance EM were purchased from Nanoprobes. Custom 3′ Biotin labeled HPLC purified oligonucleotides were purchased from Qiagen-Operon and diluted to the appropriate concentrations in 2× MES buffer. Complex tissue samples were prepared according to GeneChip® Expression Analysis Technical Manual provided by Affymetrix. Affymetrix GeneChip® Arrays (both catalog products and research tools) provided by Affymetrix. Psoralen-biotin complex purchased from Ambion (Pierce).

Instrumentation:

A Hitachi S-4700 FE-SEM was used for this work. The SEM is equipped with two Secondary electron detectors. Additional work was performed on a different Hitachi S-4700 FE-SEM equipped with a Hitachi yttrium aluminum garnet (YAG) type backscattered electron (BSE) detector. All the thin metal coatings were deposited using a Gatan Model 681 High resolution ion beam coater. The chips are processed using GeneChip® Hybridization Oven 320 and GeneChip® Fluidics Station-400 from Affymetrix and a Rotamix RKVSD from ATR. Standard calibrated laboratory equipment including pipettes and vials were also used.

Sample Preparation:

SA-Au stain: For each chip a 600 μL solution of streptavidin-gold stain. The solution includes 75 μL of Nanogold, 23 1 μL of 2× MES buffer, 24 μL BSA, 220 μL DI water and 25 μL of 5M NaCl.

Gold enhancement solution: For each chip mix 200 μL of enhancement solution. The solution is prepared following the manufacturer's instructions.

Streptavidin-gold staining: Hybridize array following an appropriate procedure for the given target. For complex targets, the chips are exposed to the target for 17 hours at 45° C. Saturation hybridizations, involving high concentrations of target (20 nM) and short hybridization times (30 min) at 45° C. were also performed. After hybridization, the array is washed using non-stringent and stringent buffers used standard washing conditions and then stained with a solution described above (BSA, MES buffer, DI water, 1.4 nm SA-Au particles, 5M NaCl) for 5 minutes. Following the staining procedure, there are additional washing steps using non-stringent buffer. Then, 200 μL of Au enhancement solution described above is then added to the array cartridge and rotated on the Rotamix at room temperature for up to 10 minutes. Enhancement results in gold particles on the order of 20-70 nanometers in diameter. Following the enhancement, the arrays are washed with DI water, removed from the cartridge and immediately dried with nitrogen. The dried array is mounted on an aluminum stub and generally coated with approximately 3 nm of Au/Pd, Pt or Cr to reduce charging. The best images were obtained at 5 kV and an emission current of 20 μA using UHR-Amode.

Each of the references mentioned above are herein incorporated by reference for all purposes as it fully set forth herein. The invention has been described with reference to various specific and preferred embodiments and techniques. However, it should be understood that many variations and modifications may be made while reviewing within the spirit and scope of the invention.

What is claimed is:

1. A method of detecting biomolecules on a microarray comprising
synthesizing said biomolecules on a microarray;
scanning said microarray with a scanning electron microscope; and
detecting said biomolecules on said microarray.

2. A method of claim 1 wherein said biomolecules are nucleotides, oligonucleotides or polynucleotides.

3. A method of claim 1 wherein said microarray is synthesized by light directed oligonucleotide synthesis.

4. A method of claim 1 wherein said method is used to detect errors in said synthesizing said biomolecules.

5. A method of claim 1 wherein said method is used to detect misalignment of said plurality of biomolecules on said microarray.

6. A method of claim 5 wherein said misalignment is detected with a resolution of less than about 5 micron.

7. A method of claim 5 wherein said misalignment is detected with a resolution of less than about 1 micron.

8. A method of claim 1 wherein said microarray is coated with a layer of metals.

9. A method analyzing interactions between a biomolecule target and a biomolecule probe on a microarray, comprising
exposing said biomolecule probe on said microarray to a plurality of biomolecule targets under a hybridization condition;
scanning said microarray with a scanning electron microscope; and
detecting said biomolecule targets binding to said biomolecule probe on said microarray.

10. A method of claim 9 wherein said microarray is synthesized by light directed syntheses.

11. A method of claim 9 wherein said biomolecules are nucleotides, oligonucleotides or polynucleotides.

12. A method of claim 9 wherein said biomolecule target is labeled with a heavy atom.

13. A method of claim 12 wherein said heavy atom is a colloidal gold.

14. A method of claim 13 wherein said heavy atom is detected using a backscattered electron detector.

15. A method of testing conditions in a microarray manufacturing process comprising
synthesizing biomolecules on a first microarray using a microarray manufacturing process with a first condition;
inspecting a pattern on said first microarray with a scanning electron microscope;
synthesizing biomolecules on a second micro array using a micro array manufacturing process with a second condition;
inspecting a pattern on said second microarray with a scanning electron microscope;
comparing said patterns on said first microarray and said second microarrays; and
selecting a condition for said microarray manufacturing process.

16. A method of claim 15 wherein said biomolecules are nucleotides, oligonucleotides or polynucleotides.

17. A method of claim 15 wherein said microarray is synthesized by light directed oligonucleotide synthesis.
